(19) United States
(12) Patent Application Publication
(10) Pub. No.: US 2019/0352594 A1
(43) Pub. Date: Nov. 21, 2019

(54) CULTURE VESSEL HOUSING APPARATUS

(71) Applicant: KATAOKA CORPORATION, Kyoto-shi (JP)

(72) Inventors: Junichi MATSUMOTO, Kyoto-shi, Kyoto (JP); Norihiro YAMAGISHI, Kyoto-shi, Kyoto (JP)

(21) Appl. No.: 16/347,788

(22) PCT Filed: Oct. 27, 2017
(86) PCT No.: PCT/JP2017/038885

§ 371 Date: May 6, 2019

(30) Foreign Application Priority Data
Nov. 10, 2016 (JP) 2016-220027

(51) Int. Cl. C12M 1/02; C12M 1/24; C12M 3/06; C12M 1/00; C12M 1/12

(52) U.S. Cl. CPC C12M 41/22; C12M 23/08; C12M 41/14; C12M 23/34; C12M 37/04; C12M 23/16

(57) ABSTRACT

In order to perform a process of emitting a laser beam toward a cell culture vessel while maintaining the atmosphere around the cell culture vessel in a desired condition, a culture vessel housing apparatus 1 is configured to include surrounding walls 312 and 322 that surround an internal space 10 in which a cell culture vessel 9 is to be housed, a transparent top panel 4 that closes an upper part of the internal space 10 surrounded by the surrounding walls 312 and 322, a heater 5 that is provided at the top panel 4 to keep the internal space 10 warm, and a transparent bottom panel 6 that closes a lower part of the internal space 10 surrounded by the surrounding walls 312 and 322 and allows a laser beam emitted toward the cell culture vessel 9 housed in the internal space 10 to pass through.

### TECHNICAL FIELD

The present invention relates to a culture vessel housing apparatus to house cell culture vessels and perform a process of emitting a laser beam toward the cell culture vessels.

### BACKGROUND ART

Recently, fast growth has been witnessed in researches and developments of regenerative therapy technology and researches in drug discovery with the use of somatic stem cells, embryonic stem cells (ES cells), and induced pluripotent stem cells (iPS cells). In these researches and developments, it is crucial to be able to produce desired target cells and tissues in a large amount with high efficiency.

The process of cell culturing normally includes subculturing, which refers to the procedure of taking a cell clump out of a cell colony that has proliferated in a culture medium and then transferring the cell clump to a fresh culture medium for another round of proliferation (See following Patent Document 1, for example).

Currently cutting a plurality of clumps out of cells which have proliferated relies on manual operation. The cutting operation takes time and work, and besides the cutting operation can cause irregularities in the size of the clumps which result in variations in the state of growth of the subcultured cells, because it is influenced by skill of operators or other individual differences.

In regenerative therapy, cell aggregates to be transplanted for replacing or regenerating damaged tissues or organs of a patient should not contain any bad or undesired cells, otherwise rightful effect may not be exerted, moreover these cells may harm the patient's health by inducing tumorigenesis, for example. However, discarding a whole culture vessel contaminated with unwanted cells decreases the yield (the rate of harvesting) of target cells or tissues, making regenerative therapy very expensive. In order to increase the yield of target cells or tissues, it is desirable to kill or remove unwanted cells present in a culture vessel and thereby avoid wasting the other cells.

Therefore attempts are being made to precisely divide cells which has proliferated into a plurality of clumps or selectively kill unwanted cells only with using laser beams exhibiting excellent condensing properties (See following Patent Document 2, for example).

Meanwhile, for culturing cells itself, CO₂ incubators (See following Patent Document 3, for example) are often used. CO₂ incubators make the internal atmosphere with a temperature of 37° C., a humidity of 100% and a carbon dioxide concentration of 5%, and keep cell culture vessels in the atmosphere. Proliferating cells create organic acids and so on lowering pH of culture mediums. In order to maintain the pH level of the culture mediums at about 7.4, sodium hydrogen carbonates are added to the culture mediums in the cell culture vessels beforehand so that hydrogen ions generated during culturing react with bicarbonate ions to form carbonic acids, and besides generating carbon dioxide from the carbonic acids balances with the carbon dioxide concentration of the atmosphere in the CO₂ incubators.

When cell culture vessels containing culture mediums to which sodium hydrogen carbonates are added are placed in the air with a low carbon dioxide concentration, the pH level of the culture mediums reaches an equilibrium value higher than 7.4. That is why it is desirable to avoid exposing the cell culture vessels to the atmosphere with a low carbon dioxide concentration to the utmost.

### RELATED ART DOCUMENTS

### Patent Documents

Patent document 1: Japanese Translation of PCT International Application Publication No. JP-T-2014-509192.

Patent document 2: Japanese Patent Application No. 2016-522839.

Patent document 3: Japanese Unexamined Patent Application Publication No. 2010-154793.

### SUMMARY OF THE INVENTION

### Problems to be Solved by the Invention

It is an object of the present invention to perform a process of emitting a laser beam toward a cell culture vessel while maintaining the atmosphere around the cell culture vessel in a desired condition.

### Means for Solving the Problems

In order to achieve the above object, a culture vessel housing apparatus according to the present invention is configured to include a surrounding wall that surrounds an internal space in which a cell culture vessel is to be housed, a transparent top panel that closes an upper part of the internal space surrounded by the surrounding wall, a heater that is provided at the top panel to keep the internal space warm, and a transparent bottom panel that closes a lower part of the internal space surrounded by the surrounding wall and allows a laser beam emitted toward the cell culture vessel housed in the internal space to pass through.

The heater is made with a transparent conducting film, for example.

It is preferable that the culture vessel housing apparatus have a gas intake to take a gas containing carbon dioxide into the internal space.

On top of that, when the bottom panel completely or practically prevents an ultraviolet light with a wavelength of about 253.7 nm from passing through, it can restrain the ultraviolet light emitted by a germicidal lamp set above the culture vessel housing apparatus from passing through the bottom panel and exerting bad influence on mechanisms or members of a laser irradiator, a microscope and so on placed below the bottom panel.

When the top panel has a double structure with a plurality of transparent plates that are spaced in a vertical direction, the heater being provided at the lower transparent plate, a high heat-retaining property for the internal space warmed by the heater can be obtained.

When the surrounding wall has a double structure with an inner wall and an outer wall, the inner wall surrounding the internal space, the outer wall being outside the inner wall and surrounding the inner wall, a higher heat-retaining property for the internal space can be obtained.

### Effects of the Invention

The present invention enables a process of emitting a laser beam toward a cell culture vessel while maintaining the atmosphere around the cell culture vessel in a desired condition.

### BRIEF DESCRIPTION OF THE DRAWINGS

FIG. 1 is a perspective view of a culture vessel housing apparatus according to the first embodiment of the present invention.

FIG. 2 is a exploded perspective view of the culture vessel housing apparatus according to the first embodiment.

FIG. 3 is a exploded perspective view of the culture vessel housing apparatus according to the first embodiment.

FIG. 4 is a cross sectional view of the culture vessel housing apparatus according to the first embodiment.

FIG. 5 is a cross sectional side view illustrating a process of emitting a laser beam according to the first embodiment.

FIG. 6 is a perspective view of a culture vessel housing apparatus according to the second embodiment of the present invention.

FIG. 7 is a exploded perspective view of the culture vessel housing apparatus according to the second embodiment.

FIG. 8 is a exploded perspective view of the culture vessel housing apparatus according to the second embodiment.

FIG. 9 is a cross sectional view of the culture vessel housing apparatus according to the second embodiment.

FIG. 10 is a cross sectional view of the culture vessel housing apparatus according to the second embodiment.

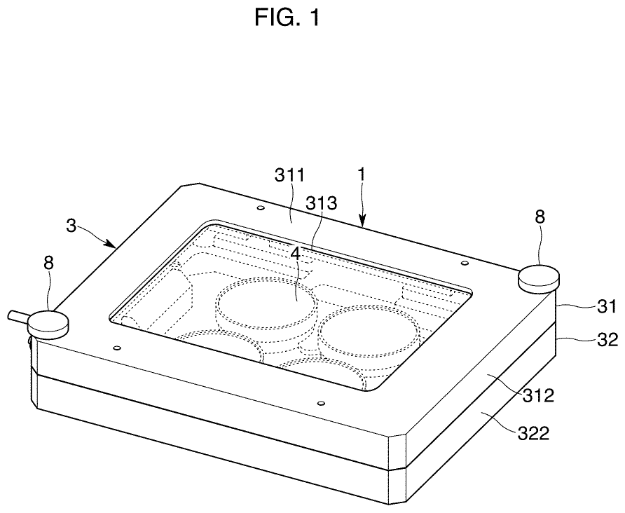

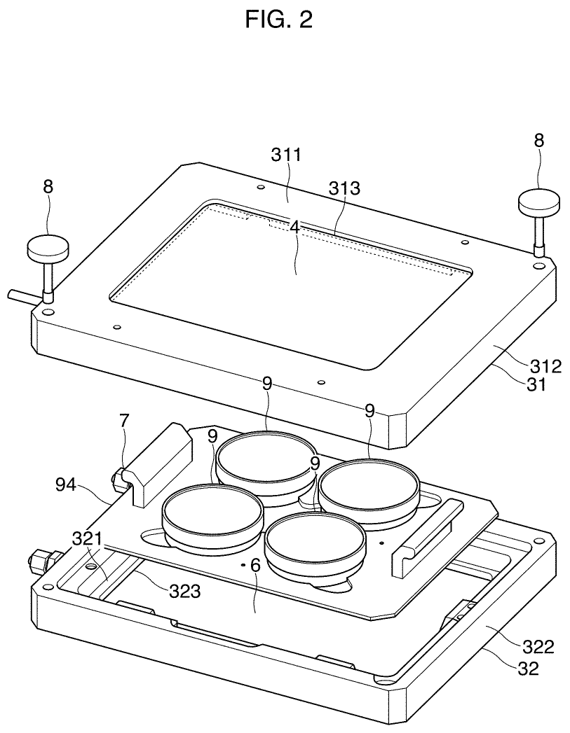

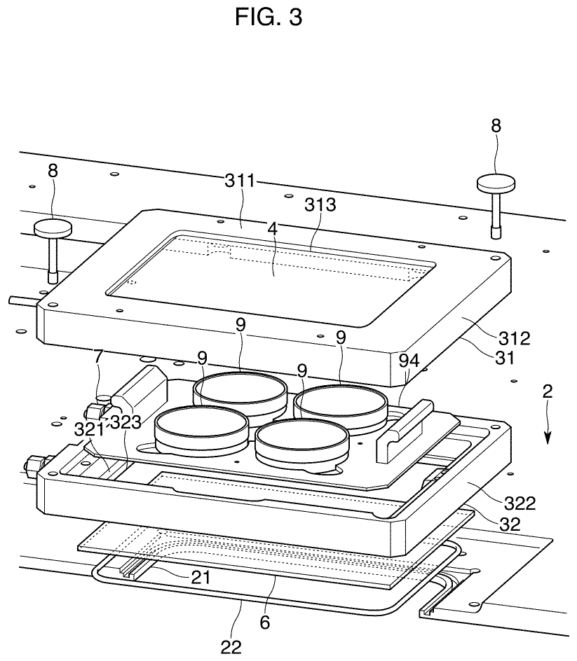

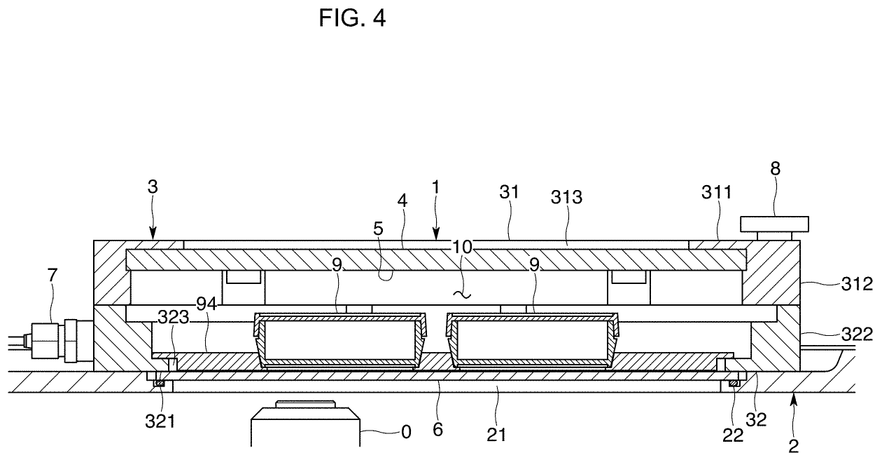

### MODE FOR CARRYING OUT THE INVENTION

<First embodiment> Described below is an embodiment of the present invention with reference to drawings. A culture vessel housing apparatus 1 according to the first embodiment shown in FIGS. 1 to 4 is put on a table 2 that divides a place (it may be an inside of a large equipment) for culturing cells, observing cells, or performing a process using a laser beam into upper and lower spaces. An atmosphere in the upper space above the table 2 is kept clean so as not to exert bad influence on cells cultured on cell culture vessels 9 or culture mediums 93. Meanwhile, a laser irradiator to emit a laser beam toward the cell culture vessels 9, a microscope to observe the cell culture vessels 9, and so on are placed in the lower space below the table 2. A window 21 that vertically pierces through the table 2 is opened in the table 2 in advance. The culture vessel housing apparatus 1 according to this embodiment is coupled onto the table 2 so as to shut the window 21 and separate the upper space from the lower space.

The culture vessel housing apparatus 1 according to this embodiment includes a frame structure 3 that forms surrounding walls 312 and 322 enclosing an internal space 10 to house the cell culture vessels 9, a top panel 4 that closes an upper part of the internal space 10 enclosed by the surrounding walls 312 and 322, a heater 5 that is provided at the top panel 4 to keep the internal space 10 warm, a bottom panel 6 that closes a lower part of the internal space 10 and allows the laser beam emitted toward the cell culture vessels 9 housed in the internal space 10 to pass through, and a gas intake 7 to take a gas containing carbon dioxide into the internal space 10.

The frame structure 3 is a metal member with a horizontally halved structure that has two components, namely, an upper frame 31 and a lower frame 32. The upper frame 31 is formed into an approximately flat box shape that opens downward, the upper frame 31 includes a top wall 311 and the surrounding wall 312 extending downward from a peripheral part of the top wall 311. An aperture 313 that vertically pierces through the top wall 311 is opened in the top wall 311. The top panel 4 is fixed to the underside of the top wall 311 and shuts the aperture 313.

The top panel 4 is transparent and is made of glass plates, acrylic plates or the like. The heater 5 is constituted by a transparent conducting film deposited on an undersurface of the top panel 4 with vapor deposition, the material of the transparent conducting film is, for example, indium tin oxide, zinc oxide. The passage of an electric current through the transparent conducting film 5 produces Joule heat that can warm the internal space 10 under the top panel 4. The heater 5 may be provided approximately all over the top panel 4, or in a limited part of the top panel 4. Also, the heater 5 may be constituted by a transparent conducting film deposited on an upper surface of the top panel 4 with vapor deposition.

The lower frame 32 is formed into an approximately flat box shape that opens upward, the lower frame 32 includes a bottom wall 321 and the surrounding wall 322 standing up on a peripheral part of the bottom wall 321. An aperture 323 that vertically pierces through the bottom wall 321 is opened in the bottom wall 321. The bottom panel 6 is set to the underside of the bottom wall 321 and shuts the aperture 323. More specifically, the bottom panel 6 closing the window 21 is put on the table 2, and besides the lower frame 32 is put thereon so that a peripheral part of the bottom panel 6 is held between the upper surface of a marginal part of the window 21 in the table 2 and the undersurface of a marginal part of the aperture 323 in the lower frame 32. An O-ring 22 is put as seal material between the marginal part of the window 21 in the table 2 and the peripheral part of the bottom panel 6.

The bottom panel 6 is transparent and is made of glass plates, acrylic plates or the like. It is preferable that the bottom panel 6 completely or practically prevent an ultraviolet light with a wavelength of 200 to 280 nm, in other words, ultraviolet C, from passing through. For this purpose, the bottom panel 6 is made of optical glass BK7 (borosilicate glass, 517642 glass), for example. Acrylic glass has the property of preventing an ultraviolet C light from passing through, too. Of course, a coating for inhibiting penetration of an ultraviolet light may be applied on the bottom panel 6. Cutting off an ultraviolet light by the bottom panel 6 is to restrain the ultraviolet light emitted by a germicidal lamp set above the table 2 with the culture vessel housing apparatus 1 from passing through the window 21 to the lower space below the table 2 and deteriorating members (paintings, plastics, rubbers molding to magnets of linear servo motors, and so on) of the laser irradiator or the microscope. On the other hand, the bottom panel 6 is transparent or light-transmissive to allow the passage of a light having a wavelength within the range of wavelength of the laser discharged from the nozzle 0 of the laser irradiator.

The upper frame 31 and the lower frame 32 are screwed together to the table 2 with screws 8. By joining the upper frame 31 to the lower frame 32 using the screws 8, the internal space 10 in the culture vessel housing apparatus 1 is isolated from the outside. In order to put the cell culture vessels 9 into or take the cell culture vessels 9 out of the internal space 10, the screws 8 are removed from the table 2, the lower frame 32 and the upper frame 31, then the upper frame 31 is detached from the lower frame 32 so as to open the internal space 10. The bottom panel 6 is detachable or exchangeable where the lower frame 32 is detached from the table 2.

The culture vessel housing apparatus 1 and the internal space 10 thereof can be cleaned with 70% ethyl alcohol aqueous solution.

The cell culture vessel 9 may be a dish (petri dish) that contains the culture mediums 93 and the cells, or a well plate that has a plurality of wells (concavities) to contain the culture mediums 93 and the cells. The cell culture vessels 9 shown in the drawings are dishes. The dishes 9 are supported on the trays 9, and besides a peripheral part of the tray 94 is engaged from above with the marginal part of the aperture 323 so that the dishes 9 and the tray 94 are supported by the lower frame 32. In this situation, a slight gap (about 1 to 2 mm) is generated between bottom faces of the dishes 9 and an upper surface of the bottom panel 6.

The nozzle 0 of the irradiator to emit the laser beam from below toward the cell culture vessels 9 or an objective lens 0 of the microscope to observe the cell culture vessels 9 is placed below the table 2 and faces the cell culture vessels 9 through the window 21 of the table 2 and the bottom panel 6. The laser beam discharged from the nozzle 0 passes through the bottom panel 6 and reaches the cell culture vessels 9. To observe the cell culture vessels by the microscope, the cell culture vessels 9 can be irradiated with illumination light that radiates from a illuminating lamp set above the culture vessel housing apparatus 1 and passes through the top panel 4 into the internal space 10.

The laser to be applied to the cell culture vessels 9 is not limited in terms of wavelength but may be a visible-light laser having such a wavelength as 405 nm, 450 nm, 520 nm, 532 nm, or 808 nm or an infrared laser, for example. It is necessary that energy of the laser having the selected wavelength be absorbed by a to-be-irradiated layer (described below) of the cell culture vessel 9.

Also, an ultraviolet laser having a wavelength of 380 nm or lower may undergo absorption by a DNA or a protein, potentially affecting cells. So, it is preferable that the wavelength of the laser be greater than 380 nm. In this embodiment, the laser source emits a continuous-wave diode laser having a wavelength near 405 nm and a maximum output of 5 W.

The nozzle 0 of the laser irradiator is equipped with, for example, a built-in lens that gathers the laser light prior to irradiation of the to-be-irradiated layer of the cell culture vessel 9 as well as a shutter or a mirror that switches between ON and OFF of the emission of the laser light. The nozzle 0 is disposed below the cell culture vessel 9 and discharges the laser upward. The optical axis of the laser beam discharged from the nozzle 0 entries into the to-be-irradiated layer of the cell culture vessel 9 at an approximately right angle. The optical system for transferring the laser from the laser source to the nozzle 0 may consist of any optical components such as optical fibers, mirrors, and lenses.

The nozzle 0 of the laser irradiator or the objective lens 0 of the microscope can be moved fast and with precision by a linear-motor sliding platform or the like in the X-axis direction (leftward and rightward) and in the Y-axis direction (frontward and backward). That is, it is possible to displace the target position on the to-be-irradiated layer of the cell culture vessel 9 where the laser is to be directed or the observation position where the objective lens 0 of the microscope captures while maintaining a substantially constant angle between the to-be-irradiated layer of the cell culture vessel 9 and the optical axis.

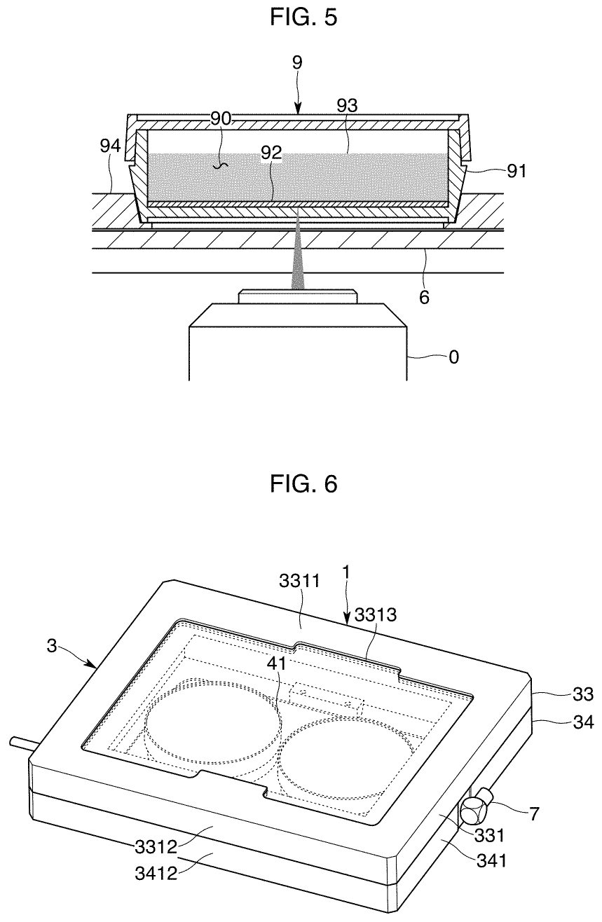

Now an additional explanation is provided with respect to the cell culture vessels 9. As shown in FIG. 5, the cell culture vessel 9 according to this embodiment comprises a main body 91 passable by the laser light discharged from the nozzle 0 and the to-be-irradiated layer 92 attached to the main body 91. The to-be-irradiated layer 92 contains a photoresponsive ingredient capable of generating heat and/or acid upon irradiation with the laser light.

The main body 91 is made of a material, such as plastic or glass, that is transparent or light-transmissive to allow the passage of a light having a wavelength within the range of wavelength of the laser discharged from the nozzle 0. Examples of the plastic include polystyrene polymers, acrylic polymers (such as poly(methyl methacrylate) (PMMA)), polyvinylpyridine polymers (such as poly(4-vinylpyridine) and 4-vinylpyridine-styrene copolymer), silicone polymers (such as polydimethylsiloxane), polyolefin polymers (such as polyethylene, polypropylene, and polymethylpentene), polyester polymers (such as poly(ethylene terephthalate) (PET) and poly(ethylene naphthalate) (PEN)), polycarbonate polymers, and epoxy polymers. The main body 91 may be a commercially-available culture vessel, which may be used as it is. In terms of shape, the main body 91 may be a dish (petri dish), a multidish, or a flask, for example, just like the shape of a commercially-available culture vessel.

The light transmittance through the main body 91 which is made of polystyrene resin is very high, as high as 85% or higher at a light wavelength of about 380 nm or greater. As the light wavelength decreases from a light wavelength of about 380 nm, the light transmittance decreases (in other words, the light absorbance by the main body 91 increases). This phenomenon is probably caused by impurities contained in the polystyrene material.

It is preferable that the to-be-irradiated layer 92 be made of a polymer (polymeric material) that contains a pigment structure (chromophore) capable of absorbing a light having a wavelength within the range of wavelength of the laser discharged from the nozzle 0. This is because such a polymer can be easily applied to the main body 91 for coating, can ensure necessary adhesion of the cells, and tends not to enter into the cells. Examples of the pigment structure capable of absorbing the laser light include derivatives of organic compounds such as azobenzene, diarylethene, spiropyrane, spirooxazines, fulgides, leucopigments, indigo, carotinoids (such as carotene), flavonoids (such as anthocyanin), and quinoids (such as anthraquinone). Examples of the polymer backbone include acrylic polymers, polystyrene polymers, polyolefin polymers, polyvinyl acetate, polyvinyl chloride, polyolefin polymers, polycarbonate polymers, and epoxy polymers.

Below is a specific example of the pigment-structure-containing polymer in the to-be-irradiated layer 92, poly[methylmethacrylate-co-(Disperse Yellow 7 methacrylate)] (Chemical Formula 1, (C₅H₈O₂)ₘ(C₂₃H₂₀N₄O₂)ₙ). The azobenzene in this azo polymer may be unsubstituted azobenzene or one of various modified azobenzenes modified with a nitro group, an amino group, and/or a methyl group.

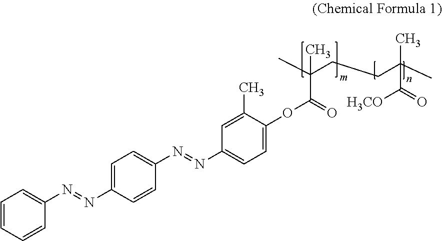

By applying a raw material liquid containing the pigment-structure-containing polymer described above or a raw material liquid containing the pigment-structure-containing polymer dissolved in a solvent (such as 1,2-dichloroethane or methanol) to the upward-facing surface of the main body 91, namely the bottom of a well 90, by spin coating, casting, or other techniques and then curing the raw material liquid, the to-be-irradiated layer 92 capable of generating heat upon irradiation with the laser light can be formed. For example, by applying a polymer containing azobenzene as the pigment structure to the upward-facing surface of the main body 91, namely the bottom of the well 90, at a density of 7 μg/cm², the to-be-irradiated layer 92 having an average thickness of 70 nm can be formed on the bottom of the well 90. Alternatively, the main body 91 may be formed by using a material blend containing a pigment capable of absorbing the laser light or by using the pigment-structure-containing polymer, and the resulting main body 91 has the function of the to-be-irradiated layer 92 capable of generating heat upon irradiation with the laser light.

The light absorbance by the to-be-irradiated layer 92 which has a certain thickness and is made by coating the main body 91 with a polymer containing azobenzene as the pigment structure reaches its peak at a light wavelength of about 360 nm and then decreases as the light wavelength increases from about 360 nm. Although the light absorbance by the to-be-irradiated layer 92 at a light wavelength of about 425 nm or greater is lower than 20%, there remains a certain level of light absorbance at great light wavelengths. This phenomenon indicates that the to-be-irradiated layer 92 is well capable of absorbing the laser light having a wavelength of 405 nm, 450 nm, 520 nm, or 532 nm.

In addition to or instead of the pigment-structure-containing polymer described above, the to-be-irradiated layer 92 may comprise a photoacid generator capable of generating an acidic substance upon irradiation with the laser light. As disclosed in Patent document 1, it is preferable that a photoacid generator contain a pigment structure (chromophore) capable of absorbing a light having a wavelength within the range of wavelength of the laser discharged from the nozzle 0 and also contain an acid precursor to be broken down into an acidic substance. Examples of the photoacid generator include sulfonic acid derivatives, carboxylic acid esters, onium salts, and photoacid-generating groups having a nitrobenzaldehyde structure.

Specific examples of the sulfonic acid derivatives as the photoacid generator include thioxanthone-based sulfonic acid derivatives (such as 1,3,6-trioxo-3,6-dihydro-1H-11-thia-azacyclopenta[a]anthracen-2-yl sulfonate) and naphthaleneimide-based sulfonic acid derivatives (such as 1,8-naphthalimide sulfonate). In addition to these, sulfonic acid derivatives such as disulfones, disulfonyldiazomethanes, disulfonylmethanes, sulfonylbenzoylmethanes, imidesulfonates, and benzoinsulfonates may also be used.

Examples of the carboxylic acid esters include 1,8-naphthalenedicarboxylic imide methylsulfonate and 1,8-naphthalenedicarboxylic imide tosyl sulfonate. Examples of the onium salts include sulfonium salts and iodonium salts containing an anion, such as tetrafluoroborate (BF₄⁻), hexafluorophosphate (PF₆⁻), and hexafluoroantimonate (SbF₆⁻).

By applying a raw material liquid containing a plastic (such as an acrylic polymer like PMMA or a polystyrene polymer, in particular) containing the photoacid generator or a raw material liquid containing the photoacid generator dissolved in a solvent (such as 1,2-dichloroethane or methanol) to the upward-facing surface of the main body 91, namely the bottom of the well 90, by spin coating, casting, or other techniques and then curing the raw material liquid, the to-be-irradiated layer 92 capable of generating heat and acid upon irradiation with the laser light can be formed. For example, by applying a polymer containing a thioxanthone-based sulfonic acid derivative having a thioxanthone backbone as the pigment structure and having a sulfonic acid as the acid precursor to the bottom of the well 90 of the main body 91 at a density of 200 μg/cm², the to-be-irradiated layer 92 having an average thickness of 2 μm can be formed on the bottom of the well 90. Alternatively, the main body 91 may be formed by using a material blend containing the photoacid generator, and the resulting main body has the function of the to-be-irradiated layer 92 capable of generating heat and acid upon irradiation with the laser light.

The light absorbance by the to-be-irradiated layer 92 which has a certain thickness and is made by coating the main body 91 with a polymer that contains a thioxanthone-based sulfonic acid derivative having a thioxanthone backbone as the pigment structure and having a sulfonic acid as the acid precursor ranges from a light wavelength of about 375 nm to a light wavelength of about 460 nm. This means that a light having a wavelength outside this range is not absorbed by the to-be-irradiated layer 92 and the laser light having a wavelength of 405 nm or 450 nm is absorbed by the to-be-irradiated layer 92. It should be noted that the light absorbance by this to-be-irradiated layer 92 is lower than the light absorbance by the to-be-irradiated layer 92 made by using a polymer that contains azobenzene as the pigment structure, and is lower than 20% (more specifically, even lower than 10%) at a visible-light wavelength ranging from about 400 nm to about 700 nm.

It is preferable that the material of the to-be-irradiated layer 92 generate no fluorescence upon irradiation with the laser light. It is preferable that the thickness of the to-be-irradiated layer 92 be 10 μm or lower.

The surface of the to-be-irradiated layer 92 of the cell culture vessel 9 may be coated with an ingredient capable of enhancing cell adhesion, such as an ECM (extracellular matrix) like laminin or Matrigel.

For culturing cells, the well 90 formed in the main body 91 of the cell culture vessel 9 is filled with a culture medium (particularly, a liquid culture medium) 93. In other words, the culture medium 93 is positioned directly on the to-be-irradiated layer 92 disposed at the bottom of the well 90. The cells thus cultured adhere to and proliferate on the surface of the to-be-irradiated layer 92 and form cell aggregates.

The laser processing for killing intended cells from among a group of cells in the well 90 in the cell culture vessel 9 is conducted in the following way. The laser light discharged from the nozzle 0 of the laser irradiator 3 is directed to a partial area of the to-be-irradiated layer 92 of the cell culture vessel 9 housed in the internal space 10 of the culture vessel housing apparatus 1 directly below the cells to be killed. In this embodiment with the nozzle 0 disposed below the cell culture vessel 9, the laser light that has been discharged upward from the nozzle 0 passes through the main body 91 to reach the to-be-irradiated layer 92 from the back side of the to-be-irradiated layer. The built-in lens in the nozzle 0 focuses or directs the laser light discharged from the nozzle 0 to the to-be-irradiated layer 92 of the cell culture vessel 9. The partial area of the to-be-irradiated layer 92 irradiated with the laser light absorbs energy of the laser light and thereby generates heat and/or acid. This heat kills unwanted cells that are present directly above the partial area.

In the case where the to-be-irradiated layer 92 comprises a photoacid generator, an acidic substance is generated in the partial area of the to-be-irradiated layer 92 irradiated with the laser light and induces death of unwanted cells present directly above the partial area or induces detachment of these cells from the to-be-irradiated layer 92. In the case where the photoacid generator is a sulfonic acid derivative, the acidic substance thus generated is a sulfonic acid.

The wavelength of the laser is 405 nm, for example. When the bottom panel 6 of the culture vessel housing apparatus 1 is made of optical glass BK7, the light transmittance of the laser with this wavelength is 92%. The output of the laser is between 0.4 W and 5 W. Of course, the output may be over 5 W. The diameter of the laser beam is less than or equal to 50 μm, for example. Of course, the diameter of the laser beam may be reduced, for example, to about 20 to 25 μm, or the diameter of the laser beam may be expanded to over 50 μm. The rate of moving the nozzle 0 discharging the continuous-wave laser or the high-frequency pulsed laser which is almost like a continuous-wave laser, or the rate of moving the laser beam, relative to the cell culture vessel 9 is set to between 50 mm/second and 2000 mm/second.

The cell culture vessels 9 in the laser processing is disposed within the culture vessel housing apparatus 1 with an internal atmosphere equivalent to that of a CO₂ incubator. The internal space 10 of the culture vessel housing apparatus 1 is supplied with a gas having a carbon dioxide concentration of 5% through the gas intake 7.

In this embodiment, the culture vessel housing apparatus 1 is configured to include the surrounding walls 312 and 322 that surround the internal space 10 in which the cell culture vessels 9 are to be housed, the transparent top panel 4 that closes the upper part of the internal space 10 surrounded by the surrounding walls 312 and 322, the heater 5 made of the transparent conducting film that is provided at the top panel 4 to keep the internal space 10 warm, the transparent bottom panel 6 that closes the lower part of the internal space 10 surrounded by the surrounding walls 312 and 322 and allows the laser beam emitted toward the cell culture vessel 9 housed in the internal space 10 to pass through, and the gas intake 7 to take the gas containing carbon dioxide into the internal space 10.

According to this embodiment, it is possible to keep the carbon dioxide concentration high while the process of emitting the laser beam toward the cell culture vessels 9 or the observation of the cell culture vessels 9 is executed, the cells cultured on the cell culture vessels 9 are not unnecessarily damaged.

In addition, according to this embodiment, as shown in FIG. 5, the laser beam is emitted from below toward the cell culture vessels 9 put in the internal space 10 of the culture vessel housing apparatus 1. The laser beam discharged from the processing nozzle 0 placed below the culture vessel housing apparatus 1 and the table 2 passes through the bottom panel 6 and is applied to the to-be-irradiated layer 92 in the cell culture vessel 9. On top of that, the heater 5 is not provided on the bottom panel 6 that the laser beam penetrates, but the heater 5 made of the transparent conducting film is laid on the top panel 4 above the cell culture vessels 9 that the laser beam does not penetrate. Such configuration makes it possible to appropriately keep the internal space 10 of the culture vessel housing apparatus 1 warm with the heater 5 even during the process of the laser irradiation. Moreover, the energy of the laser light with which the cell culture vessels 9 are irradiated is not decreased in vain due to absorbing the laser light in the transparent conducting film as the heater 5, damage to the transparent conducting film heater 5 by the laser light can be avoided. If a heater were provided on the bottom panel 6, the heater would be near to the to-be-irradiated layer 92 and be heavily damaged.

Since the bottom panel 6 completely or practically prevents an ultraviolet light with a wavelength of about 253.7 nm from passing through, it is able to avoid deteriorating the members of the laser irradiator or the microscope placed below the culture vessel housing apparatus 1 by the ultraviolet light for sterilization emitted by the germicidal lamp set above the culture vessel housing apparatus 1.

<Second embodiment> Described below is a second embodiment in which the top panel 41, 42 and the surrounding walls 3312, 3321, 3331, 3341, 3412 are configured to be a double structure each so as to improve heat retention more. Differences from the first embodiment will be mainly described hereinafter. Explanation for common points to the first embodiment is omitted.

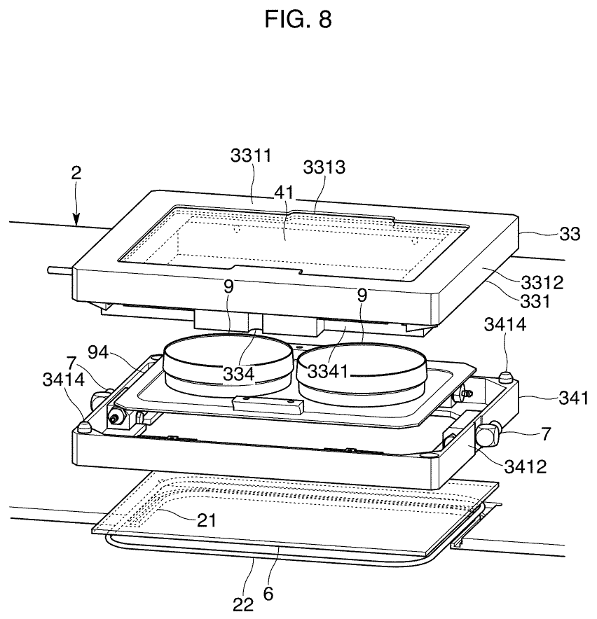

The culture vessel housing apparatus 1 according to this embodiment shown in FIGS. 6 to 10 is also put on the table 2 that divides the place for culturing the cells, observing the cells, or performing the process using the laser beam into the upper and lower spaces. The window 21 that vertically pierces through the table 2 is opened in the table 2 in advance. The culture vessel housing apparatus 1 is coupled onto the table 2 so as to shut the window 21 and separate the upper space from the lower space.

The culture vessel housing apparatus 1 according to this embodiment includes the frame structure 3 that forms the surrounding walls 3312, 3321, 3331, 3341, 3412 enclosing the internal space 10 to house the cell culture vessels 9, the top panel 41, 42 that closes the upper part of the internal space 10 enclosed by the surrounding walls 3312, 3321, 3331, 3341, 3412, the heater 5 that is provided at the top panel 4 to keep the internal space 10 warm, the bottom panel 6 that closes the lower part of the internal space 10 and allows the laser beam emitted toward the cell culture vessels 9 housed in the internal space 10 to pass through, and the gas intakes 7 to take the gas containing carbon dioxide into the internal space 10.

The frame structure 3 has the upper frame 33 and the lower frame 34. In this embodiment, the upper frame 33 has the double structure (multiple structure) with components including an outer frame 331, an inner frame 332 and an innermost frame 333, a cover frame 334 that protrudes downward from a lower end of the outer frame 331 pertains to the upper frame 33.

The outer frame 331 is formed into an approximately flat box shape that opens downward, the outer frame 331 includes a top wall 3311 and the surrounding wall 3312 extending downward from a peripheral part of the top wall 3311. The surrounding wall 3312 of the outer frame 331 is thinner than the surrounding wall 312 of the upper frame 31 in the first embodiment. An aperture 3313 that vertically pierces through the top wall 3311 is opened in the top wall 3311. The top panel 41, 42 is fixed to the underside of the top wall 3311 and shuts the aperture 3313.

The inner frame 332 is a four-side frame member with an outer periphery being approximately equal to an inner periphery of the surrounding wall 3312 of the outer frame 331, the inner frame 332 fits into the outer frame 331. A supporting piece 3322 protrudes inward and substantially horizontally from an inside surface in the vicinity of the upper end of the surrounding wall 3321 of the inner frame 332. A transparent plate 41 being the top panel is put on the upper surface of the supporting piece 3322 from above. A peripheral part of the transparent plate 41 is fixed to the supporting piece 3322.

The innermost frame 333 is a four-side frame member with an outer periphery being approximately equal to an inner periphery of the surrounding wall 3321 of the inner frame 332, the innermost frame 333 fits into the inner frame 332. A transparent plate 42 being the top panel is put on the upper surface of the surrounding wall 3331 of the innermost frame 333 from above. A peripheral part of the transparent plate 42 is fixed to the surrounding wall 3331. The transparent plate 42 and the innermost frame 333 may be integrally formed.

Each of the transparent plates 41 and 42 being the top panel is transparent and is made of glass plates, acrylic plates or the like. It goes without saying that the transparent plates 41 and 42 allow visible lights and an ultraviolet light with a wavelength of 200 to 280 nm used as a germicidal light to pass through. On that basis, the heater 5 is constituted by a transparent conducting film deposited on an upper surface and/or an undersurface of the lower transparent plate 42 facing the internal space 10 with vapor deposition. The passage of an electric current through the transparent conducting film 5 produces Joule heat that can warm the internal space 10 under the transparent plate 42. The heater 5 may be provided approximately all over the transparent plate 42, or in a limited part of the transparent plate 42.

The cover frame 334 is a four-side frame member with an outer periphery being approximately equal to an inner periphery of the surrounding wall 3412 of an outer frame 341 of the lower frame 34 that will be detailed later. The cover frame 334 fits into the outer frame 341 of the lower frame. In particular, the cover frame 334 has a role of reducing the volume of the internal space 10 to be heated by the heater 5.

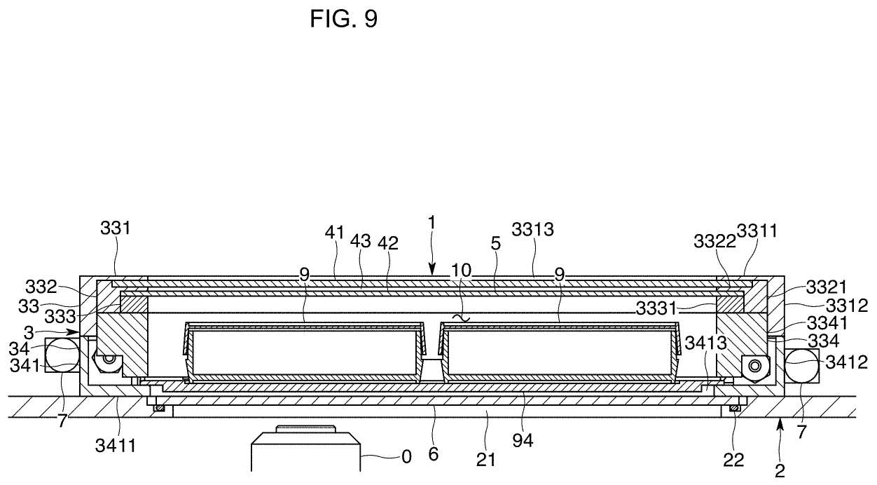

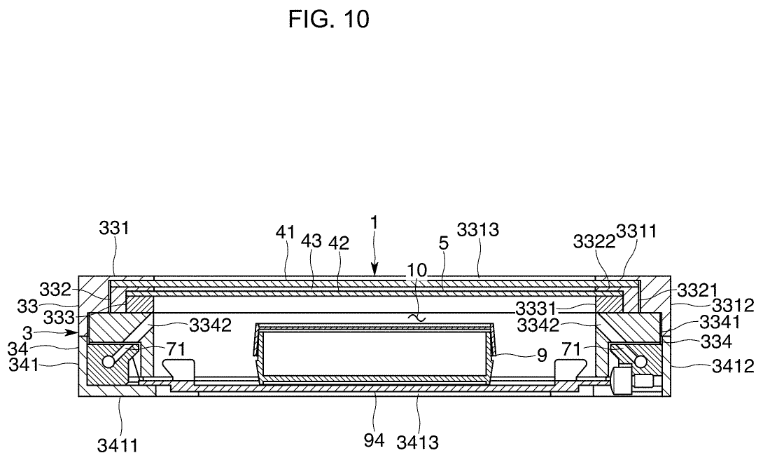

The outer frame 33, the inner frame 332 supporting the transparent plate 41, the innermost frame 333 supporting the transparent plate 42, and the cover frame 334 are combined into the upper frame 33 in the frame structure 3. Here, as shown FIGS. 9 and 10, the outside surface of the surrounding wall 3321 of the inner frame 332 is brought into contact with or in close proximity to the inside surface of the surrounding wall 3312 of the outer frame 331, the outside surface of the surrounding wall 3331 of the innermost frame 333 is brought into contact with or in close proximity to the inside surface of the surrounding wall 3321 of the inner frame 332.

In addition, the upper surface of the surrounding wall 3321 of the inner frame 332 is brought into contact with or in close proximity to the undersurface of the top wall 3311 of the outer frame 331, the upper surface of the peripheral part of the transparent plate 42 supported by the innermost frame 333 is brought into contact with or in close proximity to the undersurface of the supporting piece 3322 of the inner frame 332. Also, the transparent plates 41 and 42 are opposed to each other in a vertical direction as a gap 43 that is larger than or equal to the thickness of the supporting piece 3322 exists between those.

Further, the upper surface of the surrounding wall 3341 of the cover frame 334 is brought into contact with or in close proximity to the undersurface of the surrounding wall 3321 of the inner frame 332 and the undersurface of the surrounding wall 3331 of the innermost frame 333 in the upper frame 33.

The inner frame 332 and the innermost frame 333 may be detachable from the outer frame 331, the innermost frame 333 may be detachable from the inner frame 332. The cover frame 334 may be detachable from the inner frame 332 and the innermost frame 333.

The lower frame 34 is mainly composed of the outer frame 341 into which the cover frame 334 is inserted from above. The lower frame 34 is formed into an approximately flat box shape that opens upward, the lower frame 34 includes a bottom wall 3411 and the surrounding wall 3412 standing up on a peripheral part of the bottom wall 3411. The surrounding wall 3412 of the outer frame 341 is thinner than the surrounding wall 322 of the lower frame 32 in the first embodiment. An aperture 3413 that vertically pierces through the bottom wall 3411 is opened in the bottom wall 3411. The bottom panel 6 is set to the underside of the bottom wall 3411 and shuts the aperture 3413. More specifically, the bottom panel 6 closing the window 21 is put on the table 2, and besides the outer frame 341 of the lower frame 34 is put thereon so that the peripheral part of the bottom panel 6 is held between the upper surface of the marginal part of the window 21 in the table 2 and the undersurface of a marginal part of the aperture 3413 in the outer frame 341. The O-ring 22 is put as seal material between the marginal part of the window 21 in the table 2 and the peripheral part of the bottom panel 6.

The bottom panel 6 is transparent and is made of glass plates, acrylic plates or the like. It is preferable that the bottom panel 6 completely or practically prevent an ultraviolet light with a wavelength of 200 to 280 nm from passing through, and however, the bottom panel 6 is transparent or light-transmissive to allow the passage of a light having a wavelength within the range of wavelength of the laser discharged from the nozzle 0 of the laser irradiator.

The tray 94 to support the cell culture vessels (for example, dishes) 9 is put on the outer frame 341. More specifically, the peripheral part of the tray 94 is engaged from above with the marginal part of the aperture 3413 of the outer frame 341, thereafter the cover frame 334 is inserted from above into the outer frame 341 so as to join the upper frame 33 to the lower frame 34. Thereby, as shown in FIGS. 9 and 10, the cell culture vessels 9 supported by the tray 94 are housed inside the cover frame 334. In addition, the outside surface of the surrounding wall 3341 of the cover frame 334 is brought into contact with or in close proximity to the inside surface of the surrounding wall 3412 of the outer frame 341, the outer frame 341 and the cover frame 334 form the double structure. The undersurface of the surrounding wall 3412 of the cover frame 334 rises from the upper surface of the peripheral part of the tray 94 in a measure. A slight gap (about 1 to 2 mm) is generated between the bottom faces of the dishes 9 supported by the tray 94 and the upper surface of the bottom panel 6.

In the first embodiment, the upper frame 31 and the lower frame 32 are screwed together to the table 2 with the screws 8. In contrast, according to this embodiment, the outer frame 341 of the lower frame 34 is screwed or fixed with other means to the table 2, however, the upper frame 33 and the lower frame 34 are not screwed together. Instead, pins 3414 are provided protruding upward from the upper surface of the surrounding wall 3412 of the outer frame 341 of the lower frame 34, and besides engagement holes (not illustrated) hollow upward are formed in the undersurface of the surrounding wall 3312 of the outer frame 331 of the upper frame 33 so that the former pins 3414 are inserted into the latter engagement holes and determine the position of the upper frame 33 relative to the lower frame 34 when the lower frame 34 is covered with the upper frame 33.

By joining the upper frame 33 to the lower frame 34, the internal space 10 in the culture vessel housing apparatus 1 is isolated from the outside. Here, as shown in FIGS. 9 and 10, the undersurface of the surrounding wall 3312 of the outer frame 331 of the upper frame 33 is brought into contact with or in close proximity to the upper surface of the surrounding wall 3412 of the outer frame 341 of the lower frame 34. In order to put the cell culture vessels 9 into or take the cell culture vessels 9 out of the internal space 10, the upper frame 31 is detached from the lower frame 32 so as to open the internal space 10. In order to put the tray 94 into or take the tray 94 out of the internal space 10, the cover frame 334 in the lower frame 34 is detached from the outer frame 341 additionally. The bottom panel 6 is detachable or exchangeable where the lower frame 34 is detached from the table 2.

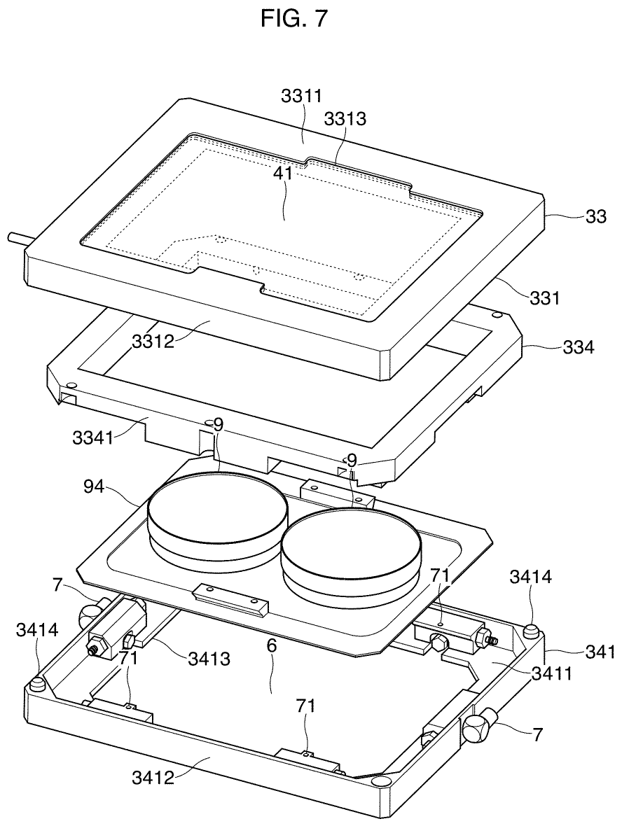

The cell culture vessels 9 in the laser processing is disposed within the culture vessel housing apparatus 1 with the internal atmosphere equivalent to that of a CO₂ incubator. The internal space 10 of the culture vessel housing apparatus 1 is supplied with the gas having a carbon dioxide concentration of 5% through the gas intakes 7. The gas jets out from jet nozzles 71 into the internal space 10. As shown in FIGS. 7 and 10, the jet nozzles 71 are provided at the front and the rear, and at plural positions that are apart from each other right and left in the internal space 10.

The jet nozzles 71 set at the front in the internal space 10 jet the gas in the rear and upper direction, particularly pointing to the heater 5 on the undersurface of the transparent plate 42. And, the jet nozzles 71 set at the rear in the internal space 10 jet the gas in the front and upper direction, particularly pointing to the heater 5 on the undersurface of the transparent plate 42. As shown in FIG. 10, if the cover frame 334 of the lower frame 34 obstructs the jet nozzle 71, a through hole 3342 will be bored in the cover frame 334 so as to link the jet nozzle 71 to the internal space 10. The through hole 3342 points to the heater 5 on the undersurface of the transparent plate 42, too.

In this embodiment, the culture vessel housing apparatus 1 is configured to include the surrounding walls 3312, 3321, 3331, 3341, 3412 that surround the internal space 10 in which the cell culture vessels 9 are to be housed, the transparent top panel 41, 42 that closes the upper part of the internal space 10 surrounded by the surrounding walls 3312, 3321, 3331, 3341, 3412, the heater 5 made of the transparent conducting film that is provided at the top panel 42 to keep the internal space 10 warm, the transparent bottom panel 6 that closes the lower part of the internal space 10 surrounded by the surrounding walls 3312, 3321, 3331, 3341, 3412 and allows the laser beam emitted toward the cell culture vessel 9 housed in the internal space 10 to pass through, and the gas intakes 7 to take the gas containing carbon dioxide into the internal space 10.

According to this embodiment, it is possible to keep the carbon dioxide concentration high while the process of emitting the laser beam toward the cell culture vessels 9 or the observation of the cell culture vessels 9 is executed, the cells cultured on the cell culture vessels 9 are not unnecessarily damaged.

According to this embodiment, similarly to the first embodiment, the laser beam is emitted from below toward the cell culture vessels 9 put in the internal space 10 of the culture vessel housing apparatus 1. The laser beam discharged from the processing nozzle 0 placed below the culture vessel housing apparatus 1 and the table 2 passes through the bottom panel 6 and is applied to the to-be-irradiated layer 92 in the cell culture vessel 9. On top of that, the heater 5 is not provided on the bottom panel 6 that the laser beam penetrates, but the heater 5 made of the transparent conducting film is laid on the top panel 4 above the cell culture vessels 9 that the laser beam does not penetrate. Such configuration makes it possible to appropriately keep the internal space 10 of the culture vessel housing apparatus 1 warm with the heater 5 even during the process of the laser irradiation. Moreover, the energy of the laser light with which the cell culture vessels 9 are irradiated is not decreased in vain due to absorbing the laser light in the transparent conducting film as the heater 5, damage to the transparent conducting film heater 5 by the laser light can be avoided.

Since the bottom panel 6 completely or practically prevents an ultraviolet light with a wavelength of about 253.7 nm from passing through, it is able to avoid deteriorating the members of the laser irradiator or the microscope placed below the culture vessel housing apparatus 1 by the ultraviolet light for sterilization emitted by the germicidal lamp set above the culture vessel housing apparatus 1.

In this embodiment, the top panel has the double structure with the plural transparent plates 41 and 42 that are spaced in a vertical direction generating the gap 43, the heater 5 is provided at the lower transparent plate 42. Hence, a high heat-retaining property for the internal space 10 warmed by the heater 5 can be obtained.

Furthermore, in this embodiment, the surrounding walls 3312, 3321, 3331, 3341, 3412 has the double structure with the inner walls 3321, 3331, 3341 and the outer walls 3312, 3412, the inner walls 3321, 3331, 3341 surround the internal space 10, the outer walls 3312, 3412 are outside the inner walls 3321, 3331, 3341 and surround the inner walls 3321, 3331, 3341. Hence, a higher heat-retaining property for the internal space 10 can be obtained.

The present invention is not limited to the above-described embodiments. Though the heaters 5 provided at the top panels 4 and 42 are made of the transparent conducting films in the above embodiments, the heaters to be provided at the top panels 4 and 42 may be made of other materials, for example, wire mesh heaters constituted by weaving wire made of copper or other metals into mesh (particularly with extremely fine wire hard to find by the naked eye of a human) so as to keep the internal space 10 in the culture vessel housing apparatus 1 warm.

The gas intakes 7 do not always take the gas containing high-concentration carbon dioxide into the internal space 10. Depending on the type of the cells cultured on the cell culture vessels 9 or the conditions of the culture mediums 93, a gas containing low-concentration carbon dioxide, a gas containing low-concentration oxygen, or other gases will be supplied to the internal space 10 through the gas intakes 7. That is, the type of the gas is determined by the desirable atmosphere around the cell culture vessels 9 to be obtained.

The upper frame 31 and the lower frame 32 are screwed together to the table 2 with the screws 8 in the first embodiment, the lower frame 34 is fixed to the table 2 with screws and so on in the second embodiment. However, fixing the upper frame 31 and the lower frames 32 and 34 to the table 2 with the screws 8 and so on is not indispensable, those may be simply put on the table 2.

Regarding to the concrete configurations of the respective components, various modifications are possible without departing from the scope and spirit of the present invention.

### DESCRIPTION OF THE REFERENCE SIGNS

1: Culture vessel housing apparatus

10: Internal space

312, 322, 3312, 3321, 3331, 3341, 3412: Surrounding wall

4, 41, 42: Top panel

43: Gap

5: Heater

52: Control unit

6: Bottom panel

7: Gas intake

0: Nozzle of laser irradiator

1. A culture vessel housing apparatus comprising:
a surrounding wall that surrounds an internal space in which a cell culture vessel is to be housed;
a transparent top panel that closes an upper part of the internal space surrounded by the surrounding wall;
a heater that is provided at the top panel to keep the internal space warm; and
a transparent bottom panel that closes a lower part of the internal space surrounded by the surrounding wall and allows a laser beam emitted toward the cell culture vessel housed in the internal space to pass through.

2. The culture vessel housing apparatus according to claim 1,
wherein the heater is made with a transparent conducting film.

3. The culture vessel housing apparatus according to claim 1,
comprising a gas intake to take a gas containing carbon dioxide into the internal space.

4. The culture vessel housing apparatus according to claim 1,
wherein the bottom panel completely or practically prevents an ultraviolet light with a wavelength of about 253.7 nm from passing through.

5. The culture vessel housing apparatus according to claim 1,
wherein the top panel has a double structure with a plurality of transparent plates that are spaced in a vertical direction, the heater being provided at the lower transparent plate.

6. The culture vessel housing apparatus according to claim 1,
wherein the surrounding wall has a double structure with an inner wall and an outer wall, the inner wall surrounding the internal space, the outer wall being outside the inner wall and surrounding the inner wall.
